(19) United States
(12) Patent Application Publication
(10) Pub. No.: US 2010/0087781 A1
(43) Pub. Date: Apr. 8, 2010

(54) MINIMALLY INVASIVE METHODS FOR THERMAL TREATMENT

(76) Inventors: Mark L. Adams, Sandy, UT (US); Michael S.H. Chu, Brookline, MA (US); Robert M. Rauker, Chester Springs, PA (US); Ashley Seehusen, Newton, MA (US); William J. Shaw, Cambridge, MA (US)

Correspondence Address: VISTA IP LAW GROUP LLP, 12930 Saratoga Avenue, Suite D-2, Saratoga, CA, 95070, US

(21) Appl. No.: 12/565,141

(22) Filed: Sep. 23, 2009

Related U.S. Application Data
(62) Division of application No. 11/336,896, filed on Jan. 23, 2006, now abandoned.

(51) Int. Cl. A61M 25/10; A61M 29/02

(52) U.S. Cl. 604/101.03; 604/101.01; 606/192; 606/27

(57) ABSTRACT

An embodiment of the invention includes inserting into a gastrointestinal lumen, a catheter having a first balloon and a second balloon spaced from the first balloon. The first balloon and the second balloon are inflated to sealingly engage the lumen. A liquid is introduced into a volume bounded by the first balloon, the second balloon and the lumen so that the liquid contacts at least a portion of the lumen.

### CROSS-REFERENCE TO RELATED APPLICATIONS

This application is a divisional of U.S. patent application Ser. No. 11/336,896, entitled “Minimally Invasive Methods for Thermal Treatment,” and filed Jan. 23, 2006, the entirety of which is incorporated herein by reference.

### BACKGROUND

The invention relates generally to medical devices for use in conjunction with a virtual colonoscopy procedure, and more particularly, to methods for thermally treating undesirable growths.

Colorectal cancer is one of the leading causes of deaths from malignancy in the United States, with only lung cancer causing more deaths annually. Colon cancer can be prevented because it usually begins as a benign polyp that grows slowly for several years before becoming cancerous. If polyps are detected and removed, the risk of developing colon cancer is significantly reduced.

Unfortunately, widespread colorectal screening and preventive efforts are hampered by several practical impediments, including limited resources, methodologic inadequacies, and poor patient acceptance leading to poor compliance. Moreover, some tests, such as the fecal occult blood test (FOBT) fail to detect the majority of cancers and pre-cancerous polyps. Additionally, since a sigmoidoscopy only examines a portion of the colon, it also misses many polyps that occur in the remainder of the colon. The accuracy of other tests, such as the barium enema, vary and are not always reliable.

A technique for detecting colorectal cancer using helical computed tomography (CT) to create computer simulated intraluminal flights through the colon was proposed as a novel approach for detecting colorectal neoplasms by Vining D J, Shifrin R Y, Grishaw E K, Liu K, Gelfand D W, Virtual colonoscopy (Abst), Radiology Scientific Prgm 1994; 193(P):446. This technique was first described by Vining et al. in an earlier abstract by Vining D J, Gelfand D W, Noninvasive colonoscopy using helical CT scanning, 3D reconstruction, and virtual reality (Abst), SGR Scientific Program, 1994. This technique, referred to as “virtual colonoscopy”, requires a cleansed colon insufflated with air, a helical CT scan of approximately 30 seconds, and specialized three-dimensional (3D) imaging software to extract and display the mucosal surface. The resulting endoluminal images generated by the CT scan are displayed to a medical practitioner for diagnostic purposes.

There have been several advances in virtual colonoscopy that have improved the imaging techniques, making it a more viable and effective screening option. One advantage of using a virtual colonoscopy as a screening process is the elimination of the invasiveness of a traditional colonoscopy. Traditional colonoscopies are preformed using a colonoscope that has a relatively large diameter (i.e., sufficient to form a seal with the anus) that includes, among other instruments, a scope, multiple lumens for introducing gas and/or liquid, and a working channel for introducing a snare or similar device into the colon. With such a device, there is a risk of straightening and/or perforating the colon because of its relative inflexibility and size.

Another advantage of the virtual colonoscopy procedure is the elimination of the preparation process associated with a traditional colonoscopy. The typical preparation process involves the use of strong laxatives to purge any fecal waste from the colon. Such a process is extremely uncomfortable and is often cited as one of the least desirable parts of the whole procedure. Complete purging is not necessary with the virtual colonoscopy procedure. Rather, a fecal contrasting agent is used to facilitate digital subtraction of any residual feces from the virtual image.

During the procedure, the patient lies on the CT scan area. A thin tube (approximately the diameter of a rectal thermometer) is placed in the rectum, through which gas is introduced into the colon. The gas is necessary to distend the bowel allowing any polyps to stand out from the normal surface. The patient holds their breath while the machine sweeps over the abdomen. The procedure is repeated with the patient lying on their stomach. The whole procedure takes approximately ten minutes.

In addition to CT scan imaging modalities, magnetic resonance imaging (MRI) can also be used to perform the virtual colonoscopy. When using MRI, only certain MRI-compatible tools can be utilized (i.e., tools with only slight ferromagnetic properties).

Even though the virtual colonoscopy is largely non-invasive as a screening process, a need still exists for non-invasive and minimally invasive devices and methods for treating the colon (e.g., removing polyps) in the event the virtual colonoscopy identifies a problem area within the colon that merits further evaluation or treatment.

For example, during conventional colonoscopies, polyps are removed using a wire-loop snare or similar device that slices the polyp from the wall of the colon. Such a technique is not effective for broad-base polyps or multiple polyps concentrated in a small area due to the excessive bleeding that could result as well as the increased risk of perforation.

What is needed is a minimally invasive method of removing polyps in the colon without the use of cutting tools such as polyp snares.

### SUMMARY OF THE INVENTION

An embodiment of the invention includes inserting into a gastrointestinal lumen, such as the colon, the upper gastrointestinal tract, the upper intestines, etc., a catheter having a first balloon and a second balloon spaced from the first balloon. The first balloon and the second balloon are inflated to sealingly engage the colon. A fluid is introduced into a volume bounded by the first balloon, the second balloon and the colon so that the fluid contacts at least a portion of the colon.

### BRIEF DESCRIPTION OF THE DRAWINGS

FIG. 1 is an illustration of a large intestine.

FIGS. 2A and 2B illustrate different types of polyps in a colon.

FIG. 3 is a schematic illustration of a system for use with an embodiment of the invention.

FIG. 4 is an illustration of an apparatus for use with an embodiment of the invention.

FIG. 4A illustrates an apparatus according to an alternative embodiment of the invention.

FIG. 5 is an illustration of an apparatus for use with another embodiment of the invention.

FIG. 5A illustrates an apparatus according to an alternative embodiment of the invention.

FIG. 6 is an illustration of an apparatus inserted in a colon according to an embodiment of the invention.

FIG. 7 is a cross-sectional schematic of an apparatus inserted in a colon according to another embodiment of the invention.

FIG. 8 is a cross-sectional view of the illustration of FIG. 7 taken along line 8-8.

FIG. 9 is a partial cut away of an apparatus inserted in a colon according to an embodiment of the invention.

FIG. 10 is a side view of an apparatus for use with a method according to an embodiment of the invention.

FIG. 11 is a side view of an apparatus for use with a method according to an embodiment of the invention.

FIG. 12 is a side view of an apparatus for use with a method according to an embodiment of the invention.

FIG. 13 is a side view of an apparatus for use with a method according to an embodiment of the invention in a first configuration.

FIG. 14 is a side view of an apparatus for use with a method according to an embodiment of the invention in a second configuration.

### DETAILED DESCRIPTION

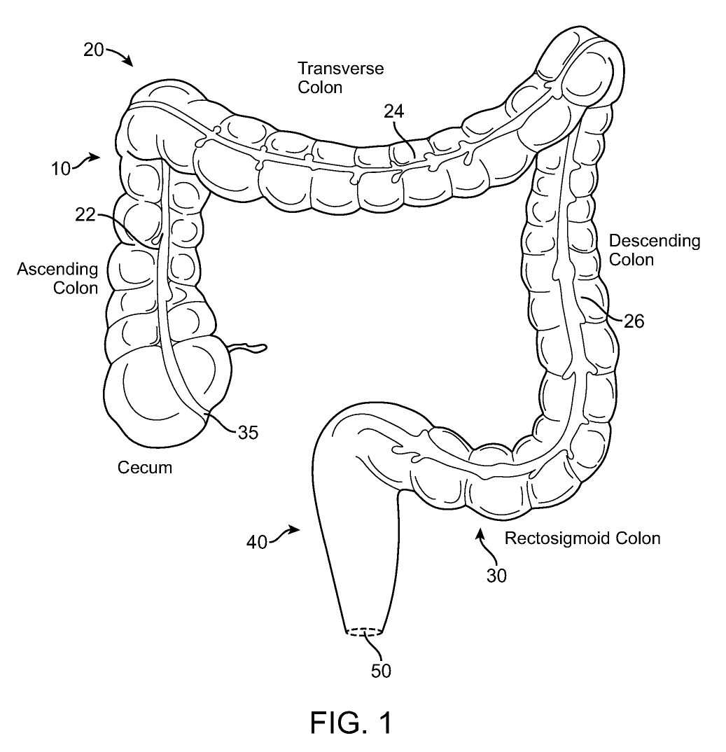

Referring to FIG. 1, an illustration of a large intestine (also called the large bowel) 10 is provided by way of background and reference. The colon 20 is the longest part of the large intestine 10, which is a tube-like organ connected to the small intestine (not illustrated) at one end and the anus 50 at the other. The colon 20 and the rectum 40 form the large intestine 10. The colon 20 is the first 4 to 5 feet of the large intestine 10, and the rectum 40 is the last 4 to 5 inches. The part of the colon 20 that joins to the rectum 40 is called the sigmoid colon 30. The junction of the two parts if often referred to as the rectosigmoid colon or rectosigmoid process. The part of the colon 20 that joins to the small intestine is called the cecum 35. The cecum 35 is adjacent the ascending colon 22, which is connected to the transverse colon 24. The transverse colon 24 is connected to the descending colon 26, which is connected to the sigmoid colon 30. The colon 20 removes/absorbs water and some nutrients and electrolytes from partially digested food. The remaining material, solid waste, called stool or feces, moves through the colon 20 to the rectum 40 and leaves the body through the anus 50.

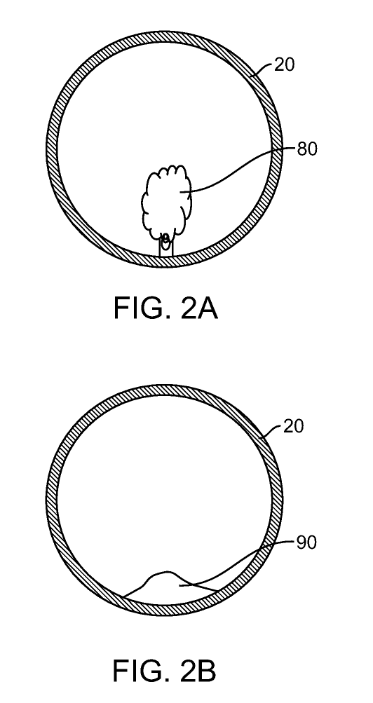

FIGS. 2A-2B illustrate various types of polyps that can form in the colon 20. A gastrointestinal polyp is a mass of the mucosal surface of the intestine that protrudes into the passageway of the bowel. Polyps can be neoplastic, non-neoplastic, or submucosal. Adenomatous polyps are abnormal growths in the colon and are more likely to develop into or already contain cancer than other types of colon polyps. Adenomatous polyps, however, usually contain tissue that is abnormal but not necessarily cancerous, hence the importance of being able to completely remove a polyp from the colon. The size, type of tissue, and degree of abnormality (mild, moderate, or severe) in a polyp determines the likelihood that it contains cancer.

Some adenomatous polyps are attached to the wall of the colon or rectum by a stalk (e.g., a pedunculated polyp 80) as illustrated in FIG. 2A. Some polyps have a broad base with little or no stalk (e.g., a sessile polyp 90) as illustrated in FIG. 2B. In some instances, several polyps are concentrated in one area of the colon.

A method of treating polyps in the colon according to an embodiment of the invention includes thermally treating the polyp using a fluid. Ideally, only the area to be treated is thermally altered. For example, the area to be treated can be fluidly isolated from the surrounding area.

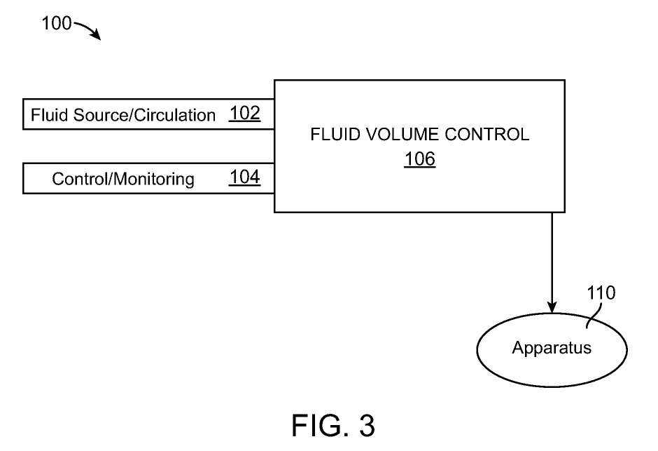

A schematic of a system for use with the method of the invention is illustrated in FIG. 3. The system 100 includes a fluid source 102 that supplies fluid to an apparatus 110 positioned within the colon 20 (not illustrated in FIG. 3). The fluid source 102 can also be configured to circulate the fluid within the apparatus 110 while the apparatus is positioned in the colon 20. For example, the fluid source 102 can circulate the fluid consistently or inconsistently. In such a configuration, the fluid source 102 can include an inspiration (i.e., injection) lumen and an aspiration (i.e., removal) lumen. The fluid source can include a heat source to heat the fluid as the fluid is circulated. A monitor 104 can measure the temperature and pressure of the fluid in the apparatus positioned in the colon. A fluid volume control 106 is configured to regulate the volume of fluid being circulated in the apparatus.

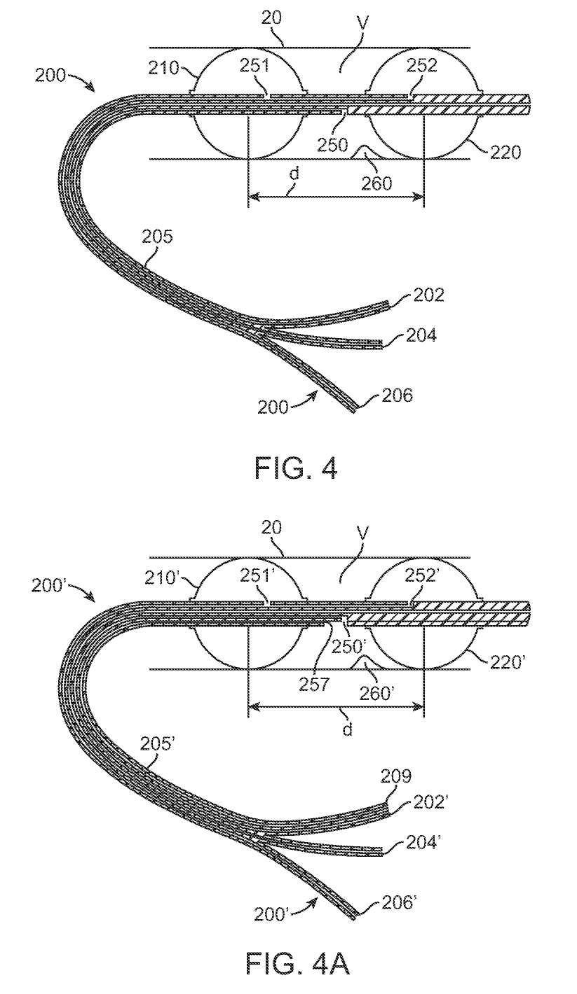

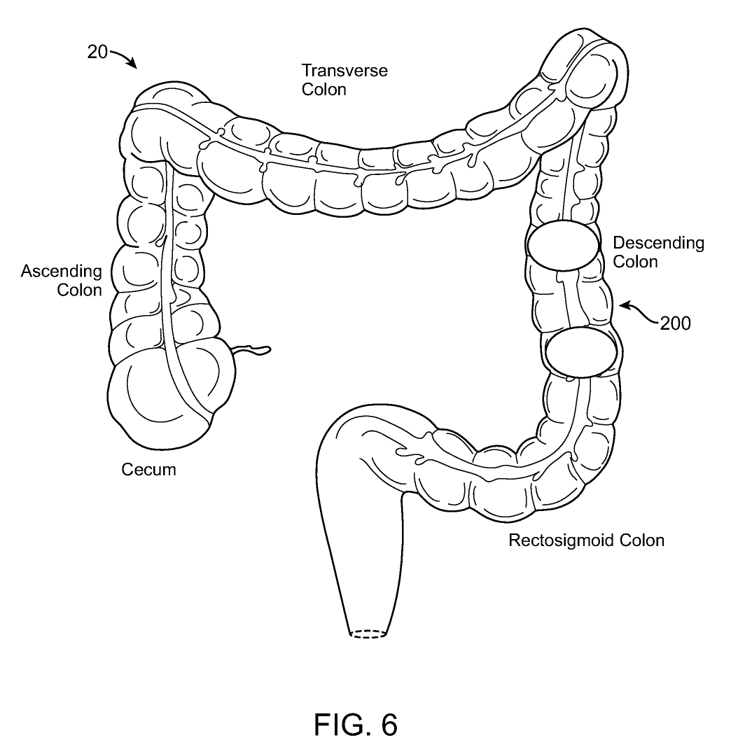

Referring to FIGS. 4 and 6, an apparatus for use with a method according to an embodiment of the invention is illustrated. A method of treating polyps in the colon includes inserting into the colon 20 via the anus 50 a catheter 200 having a tube 205, a first balloon 210 and a second balloon 220 spaced from the first balloon 210 a distance d (see FIG. 4). The first balloon 210 is inflated to a sufficient degree to sealingly engage the colon 20. The second balloon 220 is also inflated to sealingly engage the inner surface of the colon 20 at a location spaced from the location of the first balloon 210. The first balloon 210, second balloon 220 and the inner surface of the colon 20 define a bounded volume V. A fluid (not shown) is introduced into the bounded volume V so that the fluid contacts at least a portion of the colon 20. The portion of the colon 20 contacted by the fluid includes an area of interest 260 such as a polyp or group of polyps.

The fluid is introduced into the colon 20 through an opening 250 defined in the tube 205 of the catheter 200 between the first balloon 210 and the second balloon 220. The tube 205 of catheter 200 defines a first lumen 202, a second lumen 204 and third lumen 206. The first lumen 202 is configured to supply the fluid from a fluid source (not illustrated in FIG. 4) to the bounded volume V through the opening 250. The second lumen 204 is configured to supply at least one of a gas and a liquid to the colon 20. For example, an insufflation gas can be introduced to the colon 20 through the second lumen 204. In some embodiments, the second lumen 204 can be used to extract gas or liquid from the colon 20 as the first balloon 210 and the second balloon 220 are inflated.

The third lumen 206 is configured to supply an inflation medium (one or both of a pressurized gas or a liquid) to the first balloon 210 and the second balloon 220 through openings 251, 252 defined within the balloons 210, 200, respectively. The third lumen 206 can also serve as a passageway for the expulsion or extraction of the gas or liquid from the first balloon 210 and the second balloon 220.

The fluid introduced into the bounded volume can be introduced at a temperature greater than the ambient temperature in the colon 20. The temperature of the fluid contacting the portion of the colon is sufficiently high and is retained in the colon for a sufficient length of time to, for example, thermally necrose at least a portion of the colon bounding the volume V. The greater the temperature, the shorter the duration of time required to necrose the tissue to a sufficient depth to encompass the polyp(s) in area of interest 260. For example, if the fluid is heated to a temperature of up to, for example, 100° C., necrosis is essentially instantaneous at the surface of the colon (and thus the polyp) and the depth of necrosis increases relatively rapidly. Alternatively the fluid can be heated to a temperature of 50° C. and the fluid could be maintained in the colon 20 for approximately 15 minutes to achieve a depth of necrosis sufficient to necrose the polyp tissue. When lower temperatures are used for longer time periods, there is less damage to the surrounding tissue and less damage below the polyp. In some embodiments, the heat is concentrated at the mucosal level. Depending upon the fluid used, and the size of the area of interest 260, the time and temperature required to treat the area of interest 260 varies.

The fluid may be heated before or after it is introduced into the bounded volume V. For example, thermal energy can be applied to the fluid after being introduced into the bounded volume V or can be heated via an external source (not shown) before being introduced into the bounded volume V. When the treatment of the bounded volume V is completed, the fluid is removed from the bounded volume V. In some embodiments, the fluid is removed from the colon 20 after a predetermined length of time.

The fluid introduced into the bounded volume V can include, for example, water, saline, ringer's solution, gelatin, oil, polymer-based liquids, etc. The fluid introduced into the bounded volume V can also include one or more of a contrasting agent, a sclerosing agent, a necrosing agent, a therapeutic agent, a thermally activated or thermally deactivated agent and an antiangiogenic. Thus, in addition to, or instead of, providing a thermal necrosis function, the liquid can perform a chemical necrosis function, provide therapeutic benefits, provide radiographic opacity/contrast to the bounded volume V, etc. The fluid introduced into the bounded volume V need not completely fill the bounded volume V. For example, the bounded volume V need only be filled a sufficient amount to treat the area of interest 260 as will be discussed in greater detail below.

FIG. 4A illustrates an apparatus according to an alternative embodiment of the invention. The catheter 200′ includes a tube 205′, a first balloon 210′ and a second balloon 220′ spaced from the first balloon 210′ a distance d (see FIG. 4A). The first balloon 210′ is inflated to a sufficient degree to sealingly engage the colon 20. The second balloon 220′ is also inflated to sealingly engage the inner surface of the colon 20 at a location spaced from the location of the first balloon 210′. The first balloon 210′, second balloon 220′ and the inner surface of the colon 20 define a bounded volume V. A fluid (not shown) is introduced into the bounded volume V so that the fluid contacts at least a portion of the colon 20. The portion of the colon 20 contacted by the fluid includes an area of interest 260′ such as a polyp or group of polyps.

Fluid can be introduced into the colon 20 through an opening 250′ defined in the tube 205′ of the catheter 200′ between the first balloon 210′ and the second balloon 220′. Fluid can also be introduced into the colon 20 through a second opening 257 defined in the tube 205′. The tube 205′ of catheter 200′ defines a first lumen 202′, a second lumen 204′ a third lumen 206′ and a fourth lumen 209. The first lumen 202′ is configured to supply the fluid at a first temperature from a fluid source (not illustrated in FIG. 4) to the bounded volume V through the opening 250′. The second lumen 204′ is configured to supply at least one of a gas and a liquid to the colon 20. For example, an insufflation gas can be introduced to the colon 20 through the second lumen 204′. In some embodiments, the second lumen 204′ can be used to extract gas or liquid from the colon 20 as the first balloon 210′ and the second balloon 220′ are inflated.

The third lumen 206′ is configured to supply an inflation medium (one or both of a pressurized gas or a liquid) to the first balloon 210′ and the second balloon 220′ through openings 251′, 252′ defined within the balloons 210′, 200′, respectively. The third lumen 206′ can also serve as a passageway for the expulsion or extraction of the gas or liquid from the first balloon 210′ and the second balloon 220′.

The fourth lumen 209 is configured to supply fluid at a second temperature from a fluid source to the bounded volume V through the opening 257. The amounts of liquid injected through the first lumen 202′ and the fourth lumen 209 can be varied to regulate the temperature. For example, the liquid introduced through first lumen 202′ can be a hot liquid and the liquid introduced through the fourth lumen 209 can be a cold liquid. The amounts of each liquid can be varied to regulate the temperature of the liquid in the bounded volume V.

In some embodiments, the fourth lumen 209 can be used as an aspiration (i.e., removal) lumen to extract liquid from the bounded volume V. In such embodiments, liquid can be circulated within bounded volume V by supplying liquid through the first lumen 202′ and extracting liquid through the fourth lumen 209.

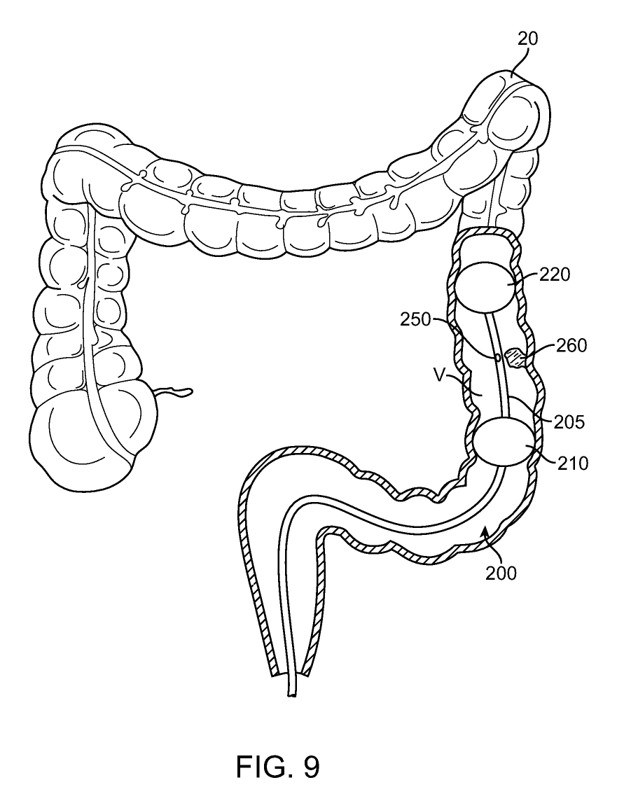

FIG. 9 is a partial cut away of an apparatus 200 inserted in a colon 20 according to an embodiment of the invention. The area of interest 260 is identified using an external imaging system such as, for example, an MRI or a CT Scan. The first balloon 210 and the second balloon 220 fluidly isolate the volume V of the colon 20 that contains the area of interest 260 from the remainder of the colon. A subsequent image of the colon may be performed to ensure proper placement of the catheter 200. Fluid is introduced into the colon 20 via opening 250 in tube 205.

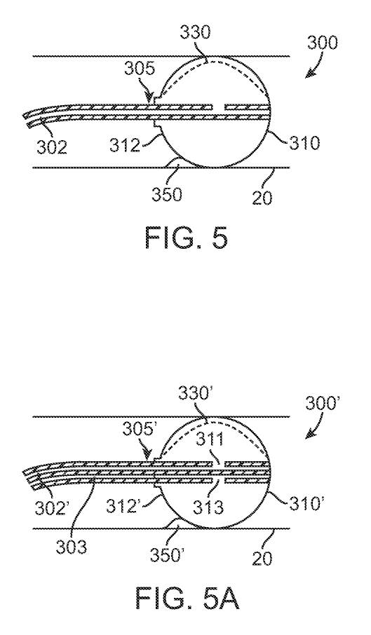

Referring to FIG. 5, an apparatus for use with a method according to another embodiment of the invention is illustrated. The method includes inserting a balloon catheter 300 having a tube 305 and at least one balloon 310 into the colon 20 via the anus 50. The balloon 310 is disposed proximate to an area of interest 350 (e.g., a polyp). The balloon 310 is expanded so that the balloon 310 engages the area of interest 350. A liquid is introduced into the balloon 310. Heat is transferred from the liquid to the area of interest via a membrane 312 that defines the volume of the balloon 310.

The balloon 310 is expanded by the liquid that is introduced into the balloon 310. Alternatively, the balloon can be expanded by a combination of the liquid introduced into the balloon 310 and a gas. Hence, the balloon 310 needs only to be filled with enough liquid such that the area of interest 350 is sufficiently heated. After a predetermined length of time, the liquid and/or gas is withdrawn from the balloon and the catheter 300 can be removed. As discussed above, the period of time can be sufficiently long and/or the temperature of the liquid can be sufficiently high to necrose the area of interest 350.

Because the area of interest 350 may only be a small portion of the colon (i.e., on one part of the wall of the colon 20), at least a portion of the membrane 312 of the balloon 310 can be insulated. In FIG. 5, the insulated portion 330 is shown in dashed lines. The insulated portion 330 can be provided by an insulating material on the balloon 310 or by making the balloon membrane 312 thicker at the insulated portion 330. Such a configuration reduces the transfer of heat to an area of the colon 20 other than the area of interest 350.

In the event the balloon 310 is not inflated such that it is in direct contact with the area of interest 350 or over the entirety of the area of interest to necrose the area of interest, the balloon 310 can be moved through the colon 20 until the area of interest 350 is sufficiently treated. As discussed above, the area of interest 350 can be identified using a virtual colonoscopy.

Referring to FIG. 5A, an apparatus according to another embodiment of the invention is illustrated. The apparatus includes a catheter 300′ having a tube 305′ and at least one balloon 310′. The balloon 310′ is disposed proximate to an area of interest 350′ (e.g., a polyp). The balloon 310′ is expanded so that the balloon 310′ engages the area of interest 350′. A liquid is introduced into the balloon 310′ via lumen 302′ though opening 311. Heat is transferred from the liquid to the area of interest via a membrane 312′ that defines the volume of the balloon 310′. A second lumen 303 can be used to introduce a second liquid into the balloon 310′ through opening 313. Alternatively, the second lumen 303 can be used to remove liquid from the balloon 310′. In this manner, liquid can readily be circulated through the balloon 310′.

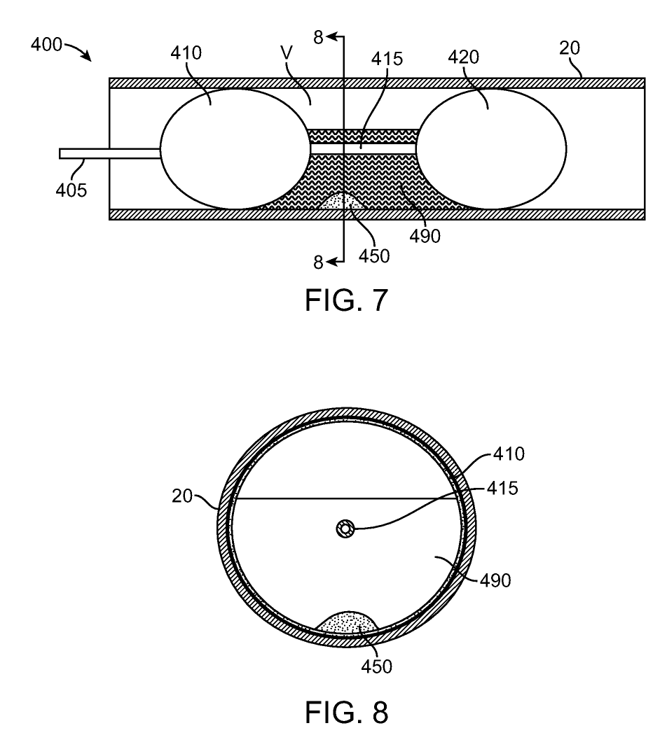

Referring to FIGS. 7 and 8, an apparatus for use with a method according to another embodiment of the invention is illustrated. A method of treating polyps in the colon includes inserting into the colon 20 via the anus 50 a catheter 400 having a tube 405, a first balloon 410 and a second balloon 420 spaced from the first balloon 410. The first balloon 410 is inflated to a sufficient degree to sealingly engage the inner surface of the colon 20. The second balloon 420 is inflated to sealingly engage the inner surface of the colon 20 at a location separate from the location of the first balloon 410. The first balloon 410, second balloon 420 and the colon 20 define a bounded volume V. A fluid 490 is introduced into the bounded volume V so that the fluid contacts at least a portion of the colon 20 at an area of interest 450.

The fluid 490 is introduced into the colon 20 through an opening (not shown) defined in the tube 405 of the catheter 400 between the first balloon 410 and the second balloon 420. The tube 405 of catheter 400 includes a heating element 415 that is configured to heat the fluid 490 introduced into the bounded volume V. A temperature sensor (not shown) may be disposed on device 400 within the bounded volume V to monitor the temperature of the fluid.

The fluid 490 introduced into the bounded volume is introduced and then heated to a predetermined temperature using heating element 415. The temperature of the fluid 490 contacting the area of interest 450 is sufficiently high and is retained in the colon for a sufficient length of time to thermally necrose at least the area of interest 450. As discussed above, the greater the temperature, the shorter the duration of time required to treat the area of interest 450.

The volume V does not need to be filled completely with fluid 490 as shown in FIGS. 8 and 9. An amount of fluid 490 sufficient to contact the area of interest 450 can be introduced into the volume V. Where a liquid is used, to ensure the area of interest 450 is contacted by the liquid 490, the patient may need to be appropriately oriented. In some embodiments, this can be accomplished by moving the patient or by using an adjustable table or chair. Such an orientation can prevent unnecessary heating of a portion of the colon 20 that does not need to be thermally treated.

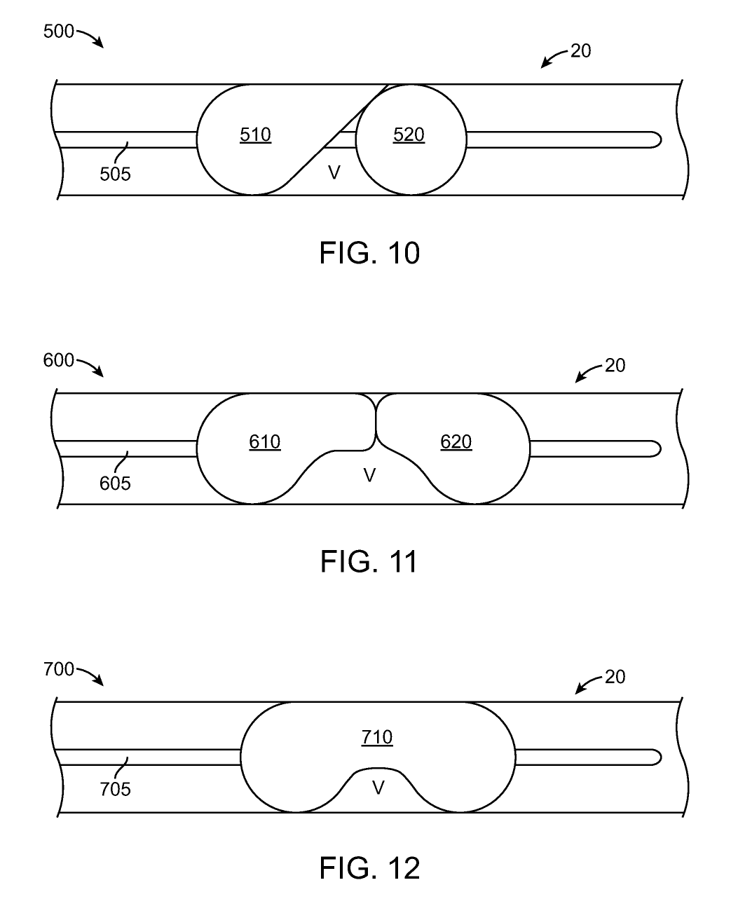

In some embodiments of the invention, balloons of various geometries are used to localize the bounded volume V. For example, referring to FIG. 10, an apparatus for use with a method according to an embodiment of the invention is illustrated. The apparatus includes a catheter 500 having a tube 505, a first balloon 510 and a second balloon 520. The first balloon 510 is inflated a sufficient degree to sealingly engage the colon. The second balloon 520 is also inflated to sealingly engage the colon. The first balloon 510 and the second balloon 520 and a portion of the inner surface of the colon define a bounded volume V. A portion of the first balloon 510 contacts the second balloon 520. When a fluid (not shown) is introduced into the bounded volume V, the fluid only engages the portion of the colon 20 that is exposed. In other words, the first balloon 510 prevents the fluid from engaging the opposite wall of the colon 20.

Referring to FIG. 11, an apparatus for use with a method according to an embodiment of the invention is illustrated. The apparatus includes a catheter 600 having a tube 605, a first balloon 610 and a second balloon 620. The first balloon 610 is inflated a sufficient degree to sealingly engage the colon. The second balloon 620 is also inflated to sealingly engage the colon. The first balloon 610 and the second balloon 620 and a portion of the inner surface of the colon define a bounded volume V. A portion of the first balloon 610 and the second balloon 620 together define a cavity between the two balloons 610, 620 and define the volume V. When a fluid (not shown) is introduced into the bounded volume V, the fluid only engages the portion of the colon 20 that is exposed. In other words, the first balloon 610 and the second balloon 620 prevent the fluid from engaging the opposite wall of the colon 20.

Referring to FIG. 12, an apparatus for use with a method according to an embodiment of the invention is illustrated. The apparatus includes a catheter 700 having a tube 705, and a balloon 710. The balloon 710 is inflated a sufficient degree to sealingly engage the colon. The balloon 710 a portion of the inner surface of the colon 20 define a bounded volume V. The balloon includes an indentation 730 that defines the bounded volume V. When a fluid (not shown) is introduced into the bounded volume V, the fluid only engages the portion of the colon 20 that is exposed. In other words, the balloon 710 prevents the fluid from engaging the opposite wall of the colon 20.

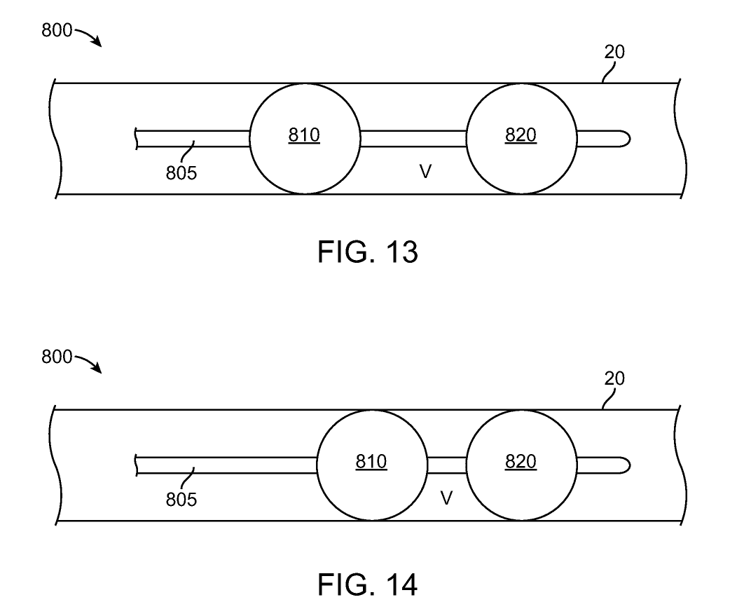

Referring to FIG. 13, an apparatus for use with a method according to an embodiment of the invention is illustrated. The apparatus includes a catheter 800 having a tube 805, a first balloon 810 and a second balloon 820. The first balloon 810 is inflated a sufficient degree to sealingly engage the colon. The second balloon 820 is also inflated to sealingly engage the colon. The first balloon 810 and the second balloon 820 and the inner surface of the colon define a bounded volume V. The balloons are slidably coupled to the tube 805 such that the size of the volume V can be modified. FIG. 13 illustrates the first balloon 810 in a first configuration and FIG. 14 illustrates the first balloon 810 in a second configuration. Either or both of the first balloon and the second balloon may be movable with respect to the tube 805.

To ensure that the balloons in each of the embodiments discussed above sealingly engage the colon 20, suction may be provided adjacent the edges of the balloons. Alternatively, the balloons can be formed from a tacky material or such a material can be applied to the outside of the balloons to promote adherence to the colon wall.

### CONCLUSION

While various embodiments of the invention have been described above, it should be understood that they have been presented by way of example only, and not limitation. Thus, the breadth and scope of the invention should not be limited by any of the above-described embodiments, but should be defined only in accordance with the following claims and their equivalents.

The previous description of the embodiments is provided to enable any person skilled in the art to make or use the invention. While the invention has been particularly shown and described with reference to embodiments thereof, it will be understood by those skilled in art that various changes in form and details may be made therein without departing from the spirit and scope of the invention.

For example, although the various balloons are shown as being spherical or oval, in alternative embodiments of the invention any shape that can fluidly isolate a volume of the colon can be used. Additionally, the balloons need not be the same shape and/or size. For example, in alternative embodiments of the invention, the balloons may be different shapes and sizes. In other embodiments, the size of the balloon can be varied to modify the size of the bounded volume within the colon.

Although described above as having one or two balloons, devices for use with the methods according to the invention may include more balloons. In some embodiments, the devices can include three balloons and more lumens in the catheter tube to provide different amounts and/or types of fluid to different areas of the colon.

Although described above as using primarily heated liquid to treat polyps, in alternative embodiments, cold liquids such as, for example, cold water or liquid nitrogen can be used to treat polyps. In further alternative embodiments, fluids of different temperatures can be introduced into the bounded volume V to regulate the temperature of the fluid in the bounded volume V. For example, hot liquid can be introduced through one lumen and cold liquid can be introduced via a different lumen. The temperature can be regulated by iteratively modifying the mixture of hot and cold liquid.

Although described above as being used to treat polyps in a colon, the methods according to the invention can be used in other channels of the body, including other gastrointestinal lumens, that can be treated using the devices described above.

What is claimed is:

1. An apparatus, comprising:
a tubular member configured to be inserted into a gastrointestinal lumen defined by a colon, the tubular member defining a first conduit and a second conduit;
a first expandable member coupled to the tubular member in fluid communication with the first conduit; and
a second expandable member coupled to the tubular member in fluid communication with the first conduit,
the first expandable member and the second expandable member configured to sealingly engage a first portion of the colon when the first expandable member and the second expandable member are each in an expanded configuration to define a volume bounded by a portion of the first expandable member, a portion of the second expandable member and a second portion of the colon, the volume in fluid communication with the second conduit, the second portion of the colon opposite at least a portion of the first portion of the colon.

2. The apparatus of claim 1, wherein the second portion of the colon is substantially diametrically opposite the portion of the first portion of the colon.

3. The apparatus of claim 1, wherein the second portion of the colon only partially surrounds the gastrointestinal lumen.

4. The apparatus of claim 1, wherein the first portion of the lumen is contiguous.

5. The apparatus of claim 1, wherein at least the first expandable member has an asymmetrical shape when in its expanded configuration.

6. The apparatus of claim 1, wherein at least the first expandable member is configured to move relative to the second expandable member such that the size of the volume can be changed.

7. The apparatus of claim 1, wherein at least the first expandable member includes a material disposed on an outer surface of the first expandable member to promote adherence of the outer surface of the first expandable member to the first portion of the colon.

8. The apparatus of claim 1, wherein:
the first expandable member is at a first position along a longitudinal axis defined by the tubular member; and
the second expandable member is at a second position along the longitudinal axis, the second position different than the first position.

9. The apparatus of claim 1, wherein the first expandable member is in contact with the second expandable member when the first expandable member and the second expandable member are each in their respective expanded configurations.

10. The apparatus of claim 1, wherein:
the volume is a first volume;
the first expandable member and a third portion of the colon define a second volume outside of and fluidically isolated from the first volume; and
the tubular member defines a third conduit configured to place the second volume in fluid communication with an area outside of the colon.

11. An apparatus, comprising:
a tubular member configured to be inserted into a gastrointestinal lumen defined by a colon, the tubular member defining a conduit;
a first expandable member coupled to the tubular member in fluid communication with the first conduit; and
a second expandable member coupled to the tubular member in fluid communication with the first conduit,
the first expandable member configured to sealingly engage a first portion of the colon when the first expandable member is in an expanded configuration, the second expandable member configured to sealingly engage a second portion of the colon when the second expandable member is in an expanded configuration such that a third portion of the colon is exposed within the gastrointestinal lumen, the third portion between the first portion and the second portion, the first portion being contiguous with the second portion.

12. The apparatus of claim 11, wherein:
the conduit is a first conduit, the tubular member defining a second conduit;
the first expandable member, the second expandable member and the third portion of the colon collectively define a volume when the first expandable member and the second expandable member are each in their respective expanded configuration, the volume in fluid communication with the second conduit.

13. The apparatus of claim 11, wherein the third portion of the colon is diametrically opposite at least a portion of the first portion of the colon or the second portion of the colon.

14. The apparatus of claim 11, wherein the first expandable member is configured to move relative to the second expandable member.

15. The apparatus of claim 11, wherein at least the first expandable member includes a material disposed on an outer surface of the first expandable member to promote adherence of the outer surface of the first expandable member to the first portion of the colon.

16. The apparatus of claim 11, wherein the first expandable member is in contact with the second expandable member when the first expandable member and the second expandable member are each in their respective expanded configurations.

17. An apparatus, comprising:
a tubular member configured to be inserted into a gastrointestinal lumen defined by a colon, the tubular member defining a first conduit and a second conduit; and
a expandable member coupled to the tubular member in fluid communication with the first conduit, the expandable member configured to sealingly engage a first portion of the colon when the expandable member is in an expanded configuration to define a first volume bounded by a portion of the expandable member and a second portion of the colon, the first volume fluidically isolated from a second volume defined within the gastrointestinal lumen distally from the first volume, the first volume in fluid communication with the second conduit, the second portion of the colon opposite at least a portion of the first portion of the colon.

18. The apparatus of claim 17, wherein:
the second portion of the colon is diametrically opposite the portion of the first portion of the colon; and
the first portion of the colon is contiguous.

19. The apparatus of claim 17, wherein the expandable member has a distal end portion, a proximal end portion and a central portion therebetween, a diameter of the central portion being less than a diameter of the distal end portion and a diameter of the proximal end portion when the expandable member is in its expanded configuration.

20. The apparatus of claim 17, wherein the expandable member includes a material disposed on an outer surface of the expandable member to promote adherence of the outer surface of the expandable member to the first portion of the colon.

21. The apparatus of claim 17, wherein the tubular member defines a third conduit configured to place the second volume in fluid communication with an area outside of the colon.
